(19) United States
(12) Patent Application Publication
(10) Pub. No.: US 2016/0206883 A1
(43) Pub. Date: Jul. 21, 2016

(54) SYSTEM AND METHOD FOR CURRENT STEERING NEUROSTIMULATION

(71) Applicant: PACESETTER, INC., Sylmar, CA (US)

(72) Inventors: Gene A. Bornzin, Simi Valley, CA (US); Lalit Venkatesan, Prosper, TX (US); Yelena Nabutovsky, Mountain View, CA (US); Riddhi Shah, San Jose, CA (US); Rupinder Bharmi, Canyon Country, CA (US); Edward Karst, Los Angeles, CA (US); Alexander Kent, Mountain View, CA (US); Xiaoyi Min, Camarillo, CA (US)

(21) Appl. No.: 14/599,960

(22) Filed: Jan. 19, 2015

(51) Int. Cl. A61N 1/36; A61N 1/05

(52) U.S. Cl. CPC A61N 1/36182; A61N 1/36178; A61N 1/0553; A61N 1/0534

(57) ABSTRACT

A system and method for current steering a neurostimulation signal is provided. The system and method provide a lead coupled to an implantable pulse generator (IPG). The lead may include a plurality of electrodes. The lead may be configured to be implanted at a target position proximate to tissue of interest. The system and method program the IPG to deliver at least a first pulse train to a first electrode and a second pulse train to a second electrode. The first and second pulse trains are interleaved with one another such that the first and second pulse trains form an activation current density distribution steered to overlay the tissue of interest.

### FIELD OF THE INVENTION

Embodiments of the present disclosure generally relate to neurostimulation (NS) systems, and more particularly to current steering stimulation signals.

### BACKGROUND OF THE INVENTION

NS systems are devices that generate electrical pulses and deliver the pulses to nervous tissue to treat a variety of disorders. For example, spinal cord stimulation has been used to treat chronic and intractable pain. Another example is deep brain stimulation, which has been used to treat movement disorders such as Parkinson's disease and affective disorders such as depression. While a precise understanding of the interaction between the applied electrical energy and the nervous tissue is not fully appreciated, it is thought that application of electrical pulses to certain regions or areas of nerve tissue can effectively mask certain types of pain transmitted from regions, increase the production of neurotransmitters, or the like. For example, applying electrical energy to the spinal cord associated with regions of the body afflicted with chronic pain can induce “paresthesia” (a subjective sensation of numbness or tingling) in the afflicted bodily regions. Thereby, paresthesia may effectively mask the transmission of non-acute pain sensations to the brain.

The effectiveness of the NS on nervous tissue may be dependent on the amplitude or frequency of the electrical pulses. Directional current steering provides the clinician with the flexibility to fine-tune the shape of the current density formed by the electrical pulses, and direct the NS to portions of the nerve tissue. However, achieving current steering using conventional systems create numerous issues. For example, maintaining charge balance on NS electrodes is important because over the life of the electrodes tens or hundreds of amp-hours may be passed, which can damage the electrodes. In another example, conventional NS systems use multiple power sources for apportioned for the electrodes increasing the size and complexity of the embedded NS system.

### SUMMARY

In accordance with one embodiment, a method for current steering a neurostimulation signal is provided. The method may provide a lead coupled to an implantable pulse generator (IPG). The lead may include a plurality of electrodes. The lead may be configured to be implanted at a target position proximate to tissue of interest. The method may program the IPG to deliver at least a first pulse train to a first electrode and a second pulse train to a second electrode. The first and second pulse trains may be interleaved with one another such that the first and second pulse trains form an activation current density distribution steered to overlay the tissue of interest.

In an embodiment, a system for current steering a neurostimulation signal is provided. The system may include a lead configured to be implanted at a target position proximate to or within a tissue of interest. The system may include a plurality of electrodes on a surface of the least, and an implantable pulse generator (IPG) coupled to the lead. The IPG may be configured to deliver at least a first pulse train to a first electrode and a second pulse train to a second electrode. The first and second pulse trains are interleaved with one another such that the first and second pulse trains form an activation current density distribution steered to overlay the tissue of interest.

### BRIEF DESCRIPTION OF THE DRAWINGS

FIG. 1 illustrates a neurostimulation system, according to an embodiment of the present disclosure.

FIG. 2 illustrates an alternative electrode configuration for a lead, according to embodiment of the present disclosure.

FIG. 3 illustrates a cross-section of the alternative electrode configuration of the lead shown in FIG. 2.

FIG. 4 illustrates a schematic diagram of the neurostimulation system, according to an embodiment of the present disclosure.

FIG. 5a illustrates a graphical representation of a pulse, according to an embodiment of the present disclosure

FIG. 5b illustrates a graphical representation of a pulse train based from the pulse in FIG. 5a, according to an embodiment of the present disclosure.

FIG. 6 illustrates a graphical representation of a series of pulse trains delivered to and emitted by two or more electrodes, according to an embodiment of the present disclosure

FIG. 7 illustrates a lead at a target position proximate to a tissue of interest, according to an embodiment of the present disclosure.

FIG. 8 illustrates a graphical representation of a series of pulse trains delivered to and emitted by two or more electrodes, according to an embodiment of the present disclosure

FIG. 9 illustrates a lead at a target position proximate to a tissue of interest, according to an embodiment of the present disclosure.

FIG. 10 is a flowchart of a method for steering a neurostimulation signal, according to an embodiment of the present disclosure.

FIG. 11 illustrates a proximate view of a portion of the lead shown in FIG. 1.

FIG. 12 illustrates a graphical representation of a series of pulse trains delivered to and emitted by two or more electrodes from the paddle structure shown in FIG. 11.

FIG. 13 illustrates a graphical representation of a series of pulse trains delivered to and emitted by two or more electrodes from the paddle structure shown in FIG. 11.

FIG. 14 illustrates a graphical representation of a series of pulse trains delivered to and emitted by two or more electrodes from the paddle structure shown in FIG. 11.

FIG. 15 illustrates a graphic of an activation area resulting from the neurostimulation signal, according to an embodiment of the present disclosure.

FIG. 16 illustrates a graphical representation of a series of pulse trains delivered to and emitted by two or more electrodes from the paddle structure shown in FIG. 11.

FIG. 17 illustrates a graphical view of an activation area resulting from the neurostimulation signal, according to an embodiment of the present disclosure.

FIG. 18 illustrates a graphical representation of a series of pulse trains delivered to and emitted by two or more electrodes from the paddle structure shown in FIG. 11.

### DETAILED DESCRIPTION

While multiple embodiments are described, still other embodiments of the described subject matter will become apparent to those skilled in the art from the following detailed description and drawings, which show and describe illustrative embodiments of disclosed inventive subject matter. As will be realized, the inventive subject matter is capable of modifications in various aspects, all without departing from the spirit and scope of the described subject matter. Accordingly, the drawings and detailed description are to be regarded as illustrative in nature and not restrictive.

Various embodiment described herein include a method and/or system to steer current to a plurality of contacts or electrodes having the same polarity towards tissue of interest. A neurostimulation (NS) signal is multiplexed and subdivided among two different electrodes. Multiplexing may be performed sufficiently fast that the energy of the NS signal is divided proportionally between the two electrodes. Based on the effective electrical properties of cells (e.g., nerve cells, neuronal cells, brain cells, brain matter) of the tissue of interest, the NS signal appears to be delivered simultaneously by the electrodes.

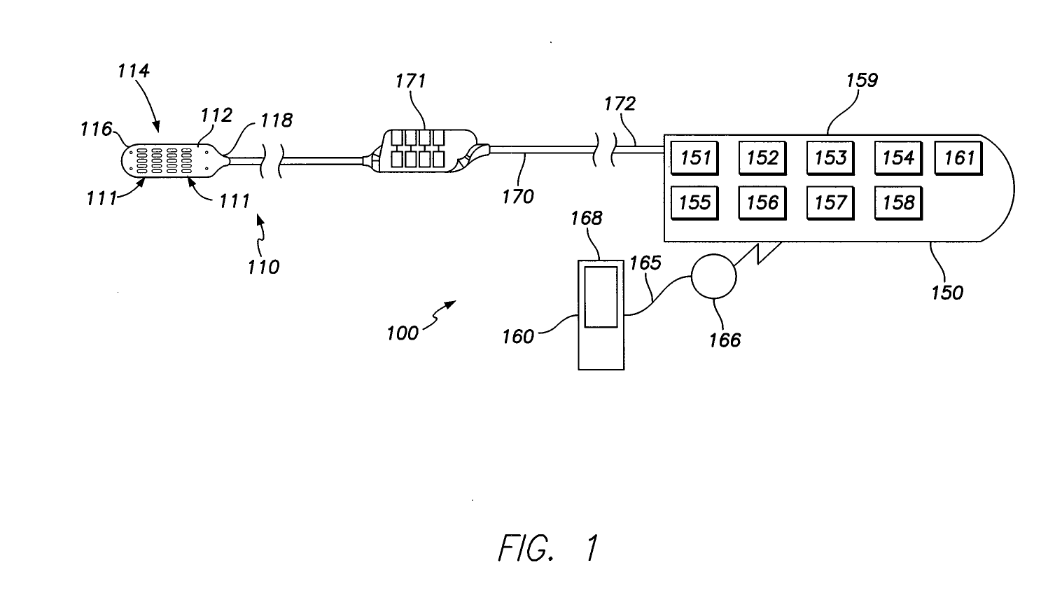

FIG. 1 depicts an NS system 100 that generates electrical pulses for application to tissue of a patient according to one embodiment. For example, the NS system 100 may be adapted to stimulate tissue of interest, such as spinal cord tissue, peripheral nerve tissue, deep brain tissue, brain matter, cortical tissue, cardiac tissue, digestive tissue, pelvic floor tissue, or any other suitable tissue of interest within a patient's body.

The NS system 100 includes an implantable pulse generator (IPG) 150 that is adapted to generate electrical pulses for application to tissue of a patient (e.g., tissue of interest). The IPG 150 typically comprises a metallic housing or can 159 that encloses a controller 151, pulse generating circuitry 152, a charging coil 153, a battery 154, a far-field and/or near field communication circuitry 155, battery charging circuitry 156, switching circuitry 157, sensing circuitry 158, memory 161, and the like. The controller 151 typically includes a microcontroller or other suitable processor for controlling the various other components of the device. Software code is typically stored in memory 261 of the IPG 150 or integrated with the controller 151 for execution by the microcontroller or processor to control the various components of the device.

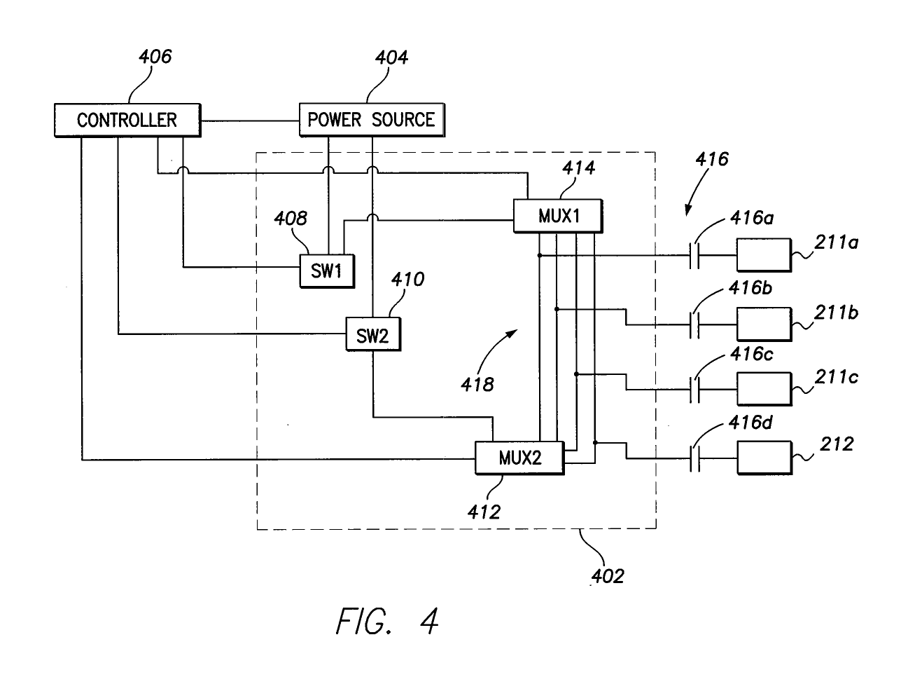

The IPG 150 may comprise a separate or an attached extension component 170. If the extension component 170 is a separate component, the extension component 170 may connect with the “header” portion of the IPG 150 as is known in the art. If the extension component 170 is integrated with the IPG 150, internal electrical connections may be made through respective conductive components. Within the IPG 150, electrical pulses are generated by the pulse generating circuitry 152 and are provided to the switching circuitry 157. The switching circuitry 157 connects to outputs of the IPG 150. Electrical connectors (e.g., “Bal-Seal” connectors) within the connector portion 171 of the extension component 170 or within the IPG header may be employed to conduct various stimulation pulses. The terminals of one or more leads 110 are inserted within connector portion 171 or within the IPG header for electrical connection with respective connectors. Thereby, the pulses originating from the IPG 150 are provided to the leads 110. The pulses are then conducted through the conductors of the lead 110 and applied to tissue of a patient via stimulation electrodes (e.g., array of electrodes 111) that may be coupled to blocking capacitors (e.g., blocking capacitors 416 in FIG. 4). Any suitable known or later developed design may be employed for connector portion 171. The lead 110 is configured to be implanted at a target position proximate to a tissue of interest (e.g., spinal cord tissue, dorsal column, within the brain, brain cells, neuronal cells, nerve tissue, or the like).

The lead 110 may include and/or be connected to a flat, thin, paddle structure 114 and connect thereto in a general longitudinal alignment with the length of the paddle structure 114. The paddle structure 114 may be formed from a medical grade, substantially inert material, for example, polyurethane, silicone, or the like. A front surface or face 112 of the paddle structure 114 is depicted in FIG. 1, which includes an array of electrodes 111 on the surface of the paddle structure 114 that are spaced apart longitudinally along the length of the paddle structure 114 from a distal end 116 and a proximal end 118. The array of electrodes 111 are spaced apart across the width of the paddle structure 114. The spacing of the electrodes 111 can be set accordingly to a target site (e.g., proximate to the spinal column, within a region of the brain) and the needed stimulation. The paddle structure 114 itself may have a width such that it spans the entire dorsal column, fits within the epidural space or target position within the brain, or the like. For example, depending upon the desired implantation site, thoracic or cervical, the paddle structure 114 may be designed to fit into the desired space such that it at least covers the anatomical and physiological midline of the patient. Additionally or alternatively, the paddle structure 114 may be similar to the paddle structure disclosed in U.S. Provisional Application No. 61/791,288, entitled, “PADDLE LEADS FOR NEUROSTIMULATION AND METHOD OF DELIVERYING THE SAME,” which is expressly incorporated herein by reference.

Each of the electrodes 111 are mutually separated by non-conducting or insulative material of the paddle, which electrically isolate each electrode 111 from adjacent electrodes 111. The non-conducting material may include one or more insulative materials and/or biocompatible materials to allow the paddle structure 114 and lead 110 to be implantable within the patient. Non-limiting examples of such materials include polyimide, polyetheretherketone (PEEK), polyethylene terephthalate (PET) film (also known as polyester or Mylar), polytetrafluoroethylene (PTFE) (e.g., Teflon), or parylene coating, polyether bloc amides, polyurethane.

The electrodes 111 may be formed of non-corrosive, highly conductive material. For example, stainless steel, MP35N, platinum, platinum alloys, or the like. The electrodes 111 may be set to function as cathodes, anodes or set to a high-impedance state for a given pulse according to the pulses generated from the IPG 150. The electrodes 111 may be configured to emit the pulses in an outward radial direction proximate to or within a stimulation target. Additionally or alternatively, the electrodes 111 may also be configured to acquire electrical potential measurements (e.g., voltage, current) or electrical signals for the sensory circuit 158, such as evoked compound activation potentials (ECAP) emitted from the stimulation target. ECAP signals may be generated by neuronal transmembrane currents of neurons activated following or in response to a stimulation pulse from one or more of the electrodes 111.

Optionally, the IPG 150 may have more than one lead 110 connected via the connector portion 171 of the extension component 170 or within the IPG header. Additionally or alternatively, the electrodes 111 of each lead 110 may be configured separately to emit current pulses or measure electrical signals emitted from and/or proximate the stimulation target.

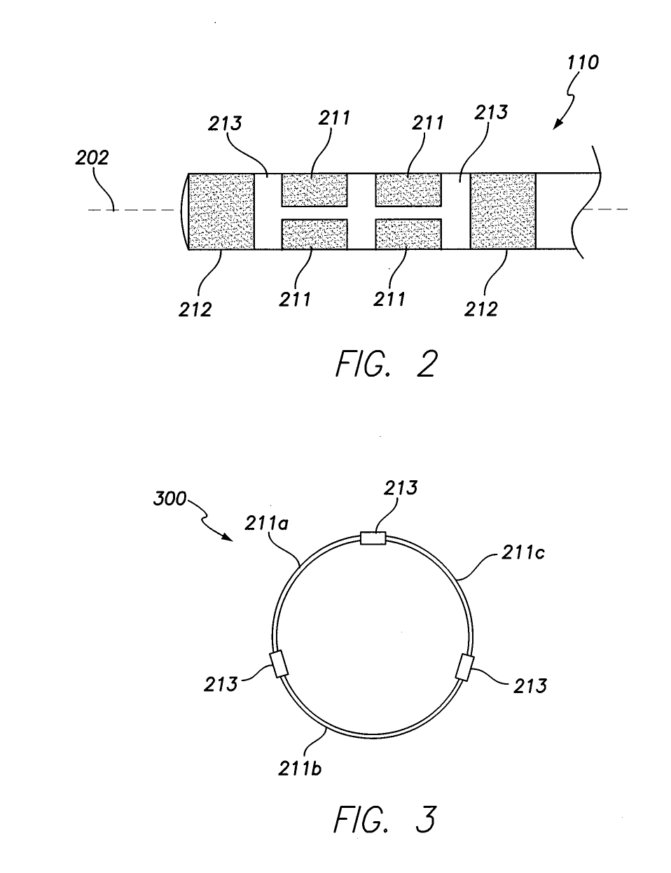

It should be noted that in other embodiments the electrodes 111 of the lead 110 may be in various other formations or structures. For example, in connection to FIGS. 2 and 3, the electrodes 111 may be in the shape of a split or non-continuous ring such as one or more segmented electrodes 211 such that the pulse may be directed in an outward radial direction adjacent to the electrodes 211. FIG. 3 illustrates a cross-section of one of the segmented electrodes 211 (e.g., 211a, 211b, 211c). Each of the segmented electrodes 211a-c are mutually separated by non-conducting or insulative material 213 of the lead 110, which electrically isolates each segmented electrode 211a-c from adjacent segmented electrodes 211a-c. The non-conducting material 213 may include one or more insulative materials and/or biocompatible materials to allow the lead 110 to be implantable within the patient.

Returning to FIG. 2, additionally or alternatively, the lead 110 may include one or more ring electrodes 212 such that each ring electrode 212 continuously covers the circumference of the exterior surface of the lead 110. The ring electrodes 212 may be configured to emit the pulses in an outward radial direction proximate to or within a stimulation target. Each of the ring electrodes 212 are separated by non-conducting material 213, which electrically isolate each ring electrode 212 from an adjacent stimulation electrode, such as the segmented electrodes 211.

The electrodes 211 and 212 may be positioned along a horizontal axis 202 of the lead 110, and are angularly positioned about the horizontal axis 202 so the electrodes 211 and 212 do not overlap. Examples of a fabrication process of the stimulation electrodes 111a-d is disclosed in U.S. Published Application Number 2011/0072657, entitled, “METHOD OF FABRICATING STIMULATION LEAD FOR APPLYING ELECTRICAL STIMULATION TO TISSUE OF A PATIENT,” which is expressly incorporated herein by reference.

Returning to FIG. 1, the lead 110 may comprise a lead body 172 of insulative material about a plurality of conductors within the material that extend from a proximal end of lead 110 (e.g., proximate to the IPG 250) to its distal end (e.g., proximate to the paddle structure 214). The conductors electrically couple a plurality of the stimulation electrodes 111 to a plurality of terminals (not shown) of the lead 110. The terminals are adapted to receive electrical pulses and the stimulation electrodes 111 are adapted to apply the pulses to the stimulation target of the patient. Also, sensing of physiological signals may occur through the stimulation electrodes 111, the conductors, and the terminals. It should be noted that although the lead 110 is depicted with a five by four array of electrodes 111, in various other embodiments, the lead 110 may include any suitable number of stimulation electrodes 111 (e.g., an array with more electrodes 111, than shown in FIG. 1, an array with less electrodes 111 than shown in FIG. 2) as well as terminals, and internal conductors. Additionally or alternatively, various sensors (e.g., a position detector, a radiopaque fiducial) may be located near the distal end 116 of the paddle structure 114 and electrically coupled to terminals through conductors within the lead body 172.

Although not required for all embodiments, the lead body 172 of the lead 110 may be fabricated to flex and elongate upon implantation or advancing within the tissue (e.g., nervous tissue) of the patient towards the stimulation target and movements of the patient during or after implantation. By fabricating the lead body 172, according to some embodiments, the lead body 172 or a portion thereof is capable of elastic elongation under relatively low stretching forces. Also, after removal of the stretching force, the lead body 172 may be capable of resuming its original length and profile. For example, the lead body may stretch 10%, 20%, 25%, 35%, or even up or above to 50% at forces of about 0.5, 1.0, and/or 2.0 pounds of stretching force. Fabrication techniques and material characteristics for “body compliant” leads are disclosed in greater detail in U.S. Provisional Patent Application No. 60/788,518, entitled “Lead Body Manufacturing,” which is expressly incorporated herein by reference.

For implementation of the components within the IPG 150, a processor and associated charge control circuitry for an IPG is described in U.S. Pat. No. 7,571,007, entitled “SYSTEMS AND METHODS FOR USE IN PULSE GENERATION,” which is expressly incorporated herein by reference. Circuitry for recharging a rechargeable battery (e.g., battery charging circuitry 156) of an IPG (e.g., the IPG 150) using inductive coupling and external charging circuits are described in U.S. Pat. No. 7,212,110, entitled “IMPLANTABLE DEVICE AND SYSTEM FOR WIRELESS COMMUNICATION,” which is expressly incorporated herein by reference.

An example and discussion of “constant current” pulse generating circuitry (e.g., pulse generating circuitry 152) is provided in U.S. Patent Publication No. 2006/0170486 entitled “PULSE GENERATOR HAVING AN EFFICIENT FRACTIONAL VOLTAGE CONVERTER AND METHOD OF USE,” which is expressly incorporated herein by reference. One or multiple sets of such circuitry may be provided within the IPG 150. Different pulses on different electrodes 111 may be generated using a single set of the pulse generating circuitry 152 using consecutively generated pulses according to a “multi-stimset program” as is known in the art. Complex pulse parameters may be employed such as those described in U.S. Pat. No. 7,228,179, entitled “Method and apparatus for providing complex tissue stimulation patterns,” and International Patent Publication Number WO 01/093953 A1, entitled “NEUROMODULATION THERAPY SYSTEM,” which are expressly incorporated herein by reference. Alternatively, multiple sets of such circuitry may be employed to provide pulse patterns (e.g., pulse trains, tonic stimulation waveform, burst stimulation waveform) that include generated and delivered stimulation pulses through various electrodes 111 of one or more leads 110 as is also known in the art. Various sets of parameters may define the pulse characteristics and pulse timing for the pulses applied to the various electrodes 211 as is known in the art. Although constant current pulse generating circuitry is contemplated for some embodiments, any other suitable type of pulse generating circuitry may be employed such as constant voltage pulse generating circuitry.

The sensing circuitry 158 may measure an electric potential (e.g., voltage, current) over time of the stimulation target or proximate tissue through at least one of the electrodes 111 that is proximate to the stimulation target and configured to measure the electrical potential. For example, the sensing circuitry 158 may measure an evoked compound action potential (ECAP) signal from an Aβ sensory fiber or nervous tissue of interest from the electric potential sensed from one or more of the electrodes 111 on the lead 110. The sensing circuitry 158 may include amplifiers, filters, analog to digital converters, memory storage devices, digital signal processors or the like. Optionally, the sensing circuitry 158 may store the electric potential in the memory 161.

A controller device 160 (e.g., an external device) may be implemented to charge/recharge the battery 154 of the IPG 150 (although a separate recharging device could alternatively be employed) and to program the IPG 150 on the pulse specifications while implanted within the patient. Although, in alternative embodiments separate programmer devices may be employed for charging and/or programming the NS system 100. The controller device 160 may be a processor-based system that possesses wireless communication capabilities. Software may be stored within a non-transitory memory of the controller device 160, which may be executed by the processor to control the various operations of the controller device 160. A “wand” 165 may be electrically connected to the controller device 160 through suitable electrical connectors (not shown). The electrical connectors may be electrically connected to a telemetry component 166 (e.g., inductor coil, RF transceiver) at the distal end of wand 165 through respective wires (not shown) allowing bi-directional communication with the IPG 150. Optionally, in some embodiments, the wand 165 may comprise one or more temperature sensors for use during charging operations.

The user may initiate communication with the IPG 150 by placing the wand 165 proximate to the NS system 100. Preferably, the placement of the wand 165 allows the telemetry system of the wand 165 to be aligned with the far-field and/or near field communication circuitry 155 of the IPG 150. The controller device 160 preferably provides one or more user interfaces 168 (e.g., graphical user interface, touchscreen, keyboard, mouse, buttons, or the like) allowing the user to operate the IPG 150. The controller device 160 may be controlled by the user (e.g., doctor, clinician) through the user interface 168 allowing the user to interact with the IPG 150. The user interface 168 may permit the user to move electrical stimulation along and/or across one or more of the lead(s) 110 using different stimulation electrode 111 combinations, for example, as described in U.S. Patent Application Publication No. 2009/0326608, entitled “METHOD OF ELECTRICALLY STIMULATING TISSUE OF A PATIENT BY SHIFTING A LOCUS OF STIMULATION AND SYSTEM EMPLOYING THE SAME,” which is expressly incorporated herein by reference. Optionally, the user interface 168, may permit the user to designate which electrodes 111 are to stimulate (e.g., emit current pulses, in an anode, state, in a cathode state) the tissue of interest, a direction of the stimulation (e.g., steering an activation current density distribution) with respect to the lead 110 and/or the tissue of interest, to measure the ECAP (e.g., from the sensing circuitry 158) resulting from the stimulation emitted by the electrodes 211, or the like.

Also, the controller device 160 may permit operation of the IPG 150 according to one or more stimulation programs to treat the patient. Each stimulation program may include one or more sets of stimulation parameters, for example that define a pulse train, including pulse amplitude, pulse width, pulse frequency or inter-pulse period, pulse repetition parameter (e.g., number of times for a given pulse to be repeated for respective stimset during execution of program), biphasic pulses, monophasic pulses, etc. The IPG 150 may modify its internal parameters in response to the control signals from the controller device 160 to vary the stimulation characteristics of the stimulation pulses transmitted through the lead 110 to the tissue of the patient, for example, in connection with steering an activation current density distribution to overlay the tissue of interest. NS systems, stimsets, and multi-stimset programs are discussed in PCT Publication No. WO 01/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are expressly incorporated herein by reference.

FIG. 4 is a basic schematic diagram of switching circuitry 402 for an embodiment of an NS system (e.g., the NS system 100). The switching circuitry 402 (e.g., the switching circuitry 157) may be electrically coupled to a controller 406 (e.g., the controller 151), a power source 404 (e.g., battery 154), and a plurality of blocking capacitors 416a-d. The switching circuitry 402 is shown with two electrical switches, a switch1 408 and a switch2 410. The switches 408 and 410 are electrically coupled to two multiplexers, a MUX1 414 and a MUX2 412. It should be noted that the switching circuitry 402 may be generally characterized as switch arrays (e.g., plurality FETS, relays), switch matrixes, or the like. Thereby, in alternative embodiments the switching circuitry 402 may include more or less switches (e.g., more than two, less than two) than illustrated in FIG. 4. Additionally, the switching circuitry may include more or less multiplexers (e.g., more than two, less than two) than illustrated in FIG. 4. Optionally the switching circuitry 402 may be integrated within the controller 406. Optionally, the switching circuitry 402 (FIG. 4) may be electrically coupled to a can (e.g., the can 159).

The switch1 408 and switch2 410 are electrically coupled to a power source 404 (e.g., the battery 154, boost converter). The power source 404 provides a direct current or voltage contact for the switch1 408 and switch2 410. The switch1 408 and switch2 410 are also electrically coupled to a common ground (not shown) for the NS system. The common ground provides a return path for electric current for the NS system. The switch1 408 and switch2 410 may direct current or form electrical current paths from the power source 404 and/or the common ground to the multiplexers 412 and 414 by electrically coupling to one of the contacts (e.g., the power source 404, the common ground). For example, the switch1 408 may electrically couple the power source 404 to the MUX1 414 and the switch2 410 may electrically couple the MUX2 412 to the common ground. Thereby, the MUX 414 may receive current or voltage from the power source 404.

The multiplexers 412 and 414 are each electrically coupled to a plurality of blocking capacitors 416 (e.g., 416a-d) through conducting paths or wires 418. Each blocking capacitor 416a-d is coupled to a corresponding electrode, such as the segmented electrodes 211a-c and the ring electrode 212. The multiplexers 412 and 414 each may select or electrically couple one or more of the blocking capacitors 416a-d to the switches 408 and 410. Continuing from the above example configuration of switch1 408 and switch2 410, MUX1 414 selects the blocking capacitor 416d and MUX2 412 selects the blocking capacitor 416a. Thereby, the blocking capacitor 416d is electrically coupled to the power source 404, and the blocking capacitor 416a is electrically coupled to the common ground. It should be understood that the multiplexers 412 and 414 may select multiple (e.g., more than one) blocking capacitors 416a-d.

Additionally or alternatively, the multiplexers 412 and 414 may be positioned between the electrodes 211a-c and 212 and the blocking capacitors 416 reducing a number of blocking capacitors 416 relative to the number shown in FIG. 4. For example, a pair of blocking capacitors 416 may be coupled to a corresponding switch 408 and 410. Each multiplexer 412 and 414 may be coupled to a corresponding blocking capacitor 416, thereby the multiplexers 412 and 414 are electrically coupled to one of the corresponding switches 408 and 410 and the electrodes (e.g., the segmented electrodes 211a-c, the ring electrode 212).

The switching circuitry 402 and the power source 404 are controlled by the controller 406 to configure pulses that are emitted from the NS system through the electrodes 211a-c, 212. The controller 406 controls or adjust the amount of current or voltage supplied to the switches 408 and 410 by instructing the amount of current or voltage supplied by the power source 404 to the switches 408 and 410. Additionally, the controller 406 may instruct at least one of the switches 408 and 410 to electrically couple to one of the multiplexers 412 and 414. Likewise, the controller 406 may instruct the multiplexers 412 and 414 to select at least one of the blocking capacitors 416a-d.

For example, the NS system 100 is programmed to emit a 2 milli-ampere (mA) pulse. The pulse is programmed to be discharged from the segmented electrode 211a in an anode state or when the stimulation electrode 211a is electrically coupled to the power source 404 via the MUX1 414, and grounded by the ring electrode 212 in a cathode state or when the ring electrode 212 is electrically coupled to the common ground via the MUX2 412. The controller 406 may instruct the power source 404 to supply a 2 mA supply current to the switches 408 and 410. The controller 406 may instruct the switch1 408 to direct current or electrically couple the MUX1 414 to the power source 404, and have the MUX1 414 select the blocking capacitor 416a that is coupled to the stimulation electrode 211a. The controller 406 may further instruct the switch2 410 to electrically couple the MUX2 412 to the common ground, and have the MUX2 412 select the blocking capacitor 416d that is coupled to the stimulation electrode 212.

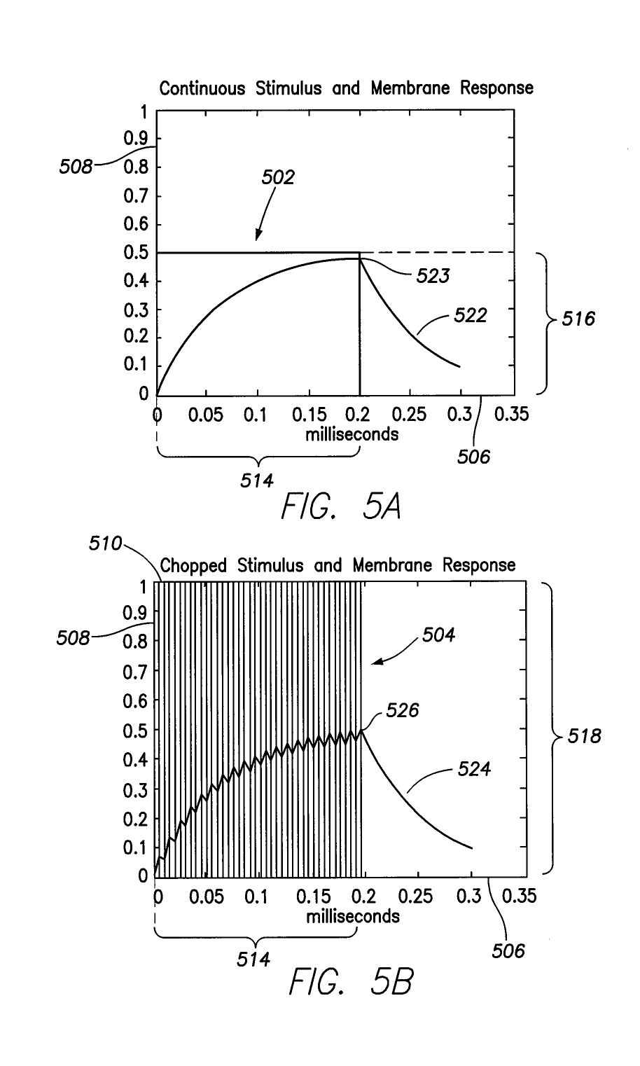

It should be noted that prior to excitation of the cell from the tissue of interest, the electrical responses of the membrane of the cells of the tissue of interest behaves similarly to a low-pass filter, which is described further below in regard to FIGS. 5a-b. For example, FIG. 5a illustrates a pulse 502 with a pulse width 514 and amplitude 516. The horizontal axes 506 represent time and the vertical axes 508 may represent current or voltage. The pulse 502 is subdivided into a series of pulses 510 forming a pulse train 504, as shown in FIG. 5b, such that over the length of time of the pulse width 514 the series of pulses 510 have a duty cycle of 50%. It should be noted that an amplitude 518 of the series of pulses 510 are twice the amplitude 518 of the pulse 502. The increased amplitude is due to the duty cycle of the pulses 510 and the electrical response of the cell membrane of the cell (e.g., nerve cells, neurons, brain matter, brain cells) (e.g., 512 and 520) receiving the stimulation.

The electrical response 512 of the cell to the pulse 502 is shown in FIG. 5a having an exponential increase in charge during the pulse 502 and repolarization 522, beginning at 523, once the cell is stimulated, after the pulse 502. The electrical response 520 of the cell to the pulse train 504 is shown in FIG. 5b. During the pulse train 504, the membrane of the cell integrates the series of pulses 510, similar to a low pass filter, until the cell is stimulated at 526 and repolarization occurs, at a similar rate as the repolarization 522. Due to the integration of the cell membrane to the pulse train 504 other possible combinations of pulse train 504 duty cycles and amplitudes 518 may be used in alternative embodiments (e.g., 80% duty cycle having an amplitude 1.25 times the amplitude 616, 66% duty cycle having an amplitude 1.5 times the amplitude 616, 33% duty cycle having an amplitude 3 time the amplitude 616, 20% duty cycle having an amplitude 5 times the amplitude 616).

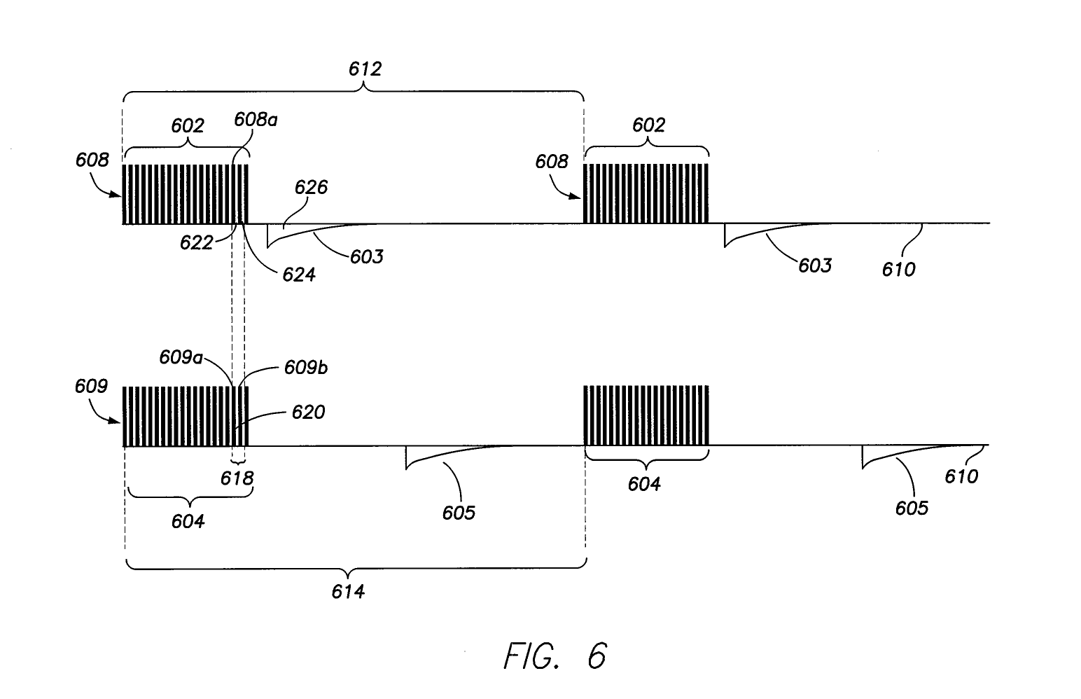

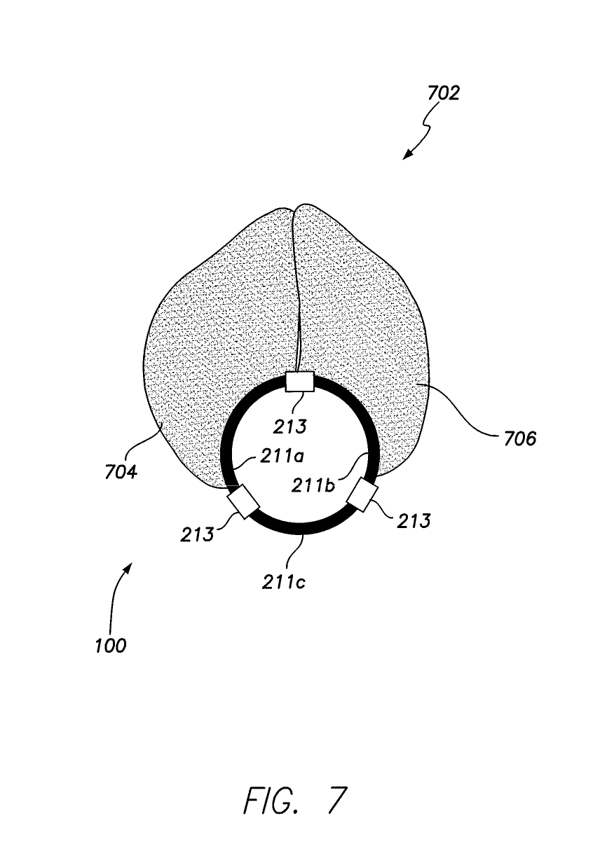

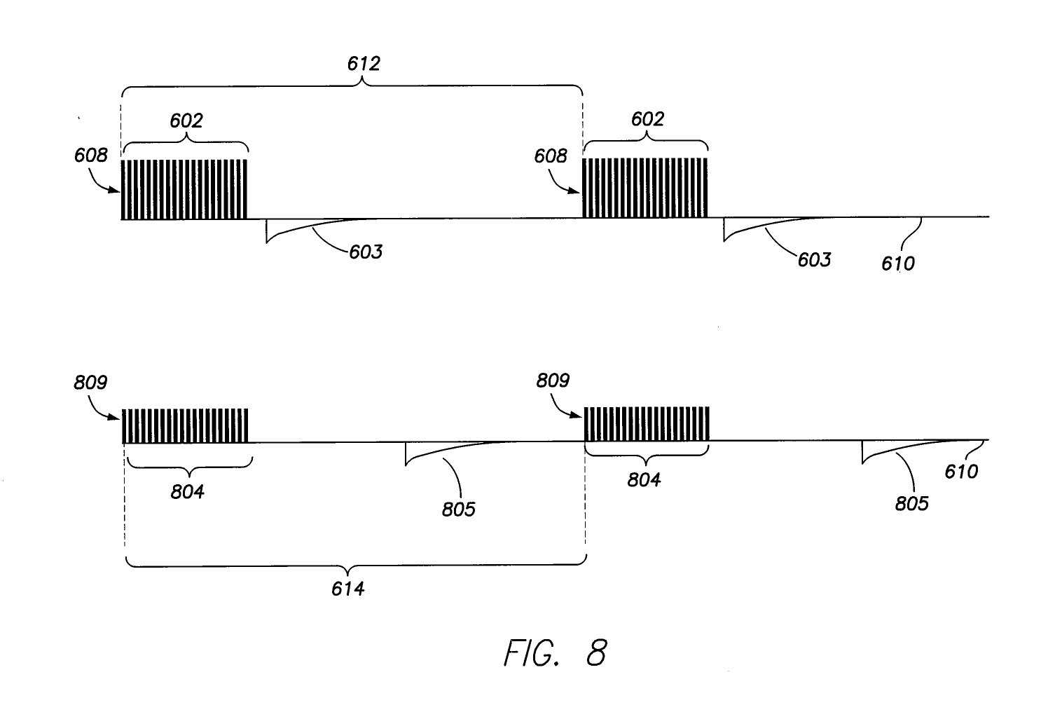

FIG. 6 illustrates graphical representations of a series of pulse trains 602, 604 delivered to and emitted by the segmented electrodes 211a, 211b, respectively. The pulse trains 602, 604 may be delivered by the IPG 150 and have the same polarity. The horizontal axes 610 represents time. Each of the pulse trains 602, 604 may correspond to two current pulses, which have been time multiplexed with each other, such that, the current pulses are subdivided into a series of pulses 608 that are interleaved in a multiplexed manner forming the first pulse train 602 and a series of pulses 609 forming the second pulse train 604. It should be noted that although the series of pulses 608, 609 shown in FIG. 6 have the same amplitudes, in various other embodiments the amplitudes of the series of pulses 608, 609 may increase, decrease, and/or change at a constant or variable rate during the pulse train 602, 604 such that at least one of the pulses 608, 609 of one or both of the pulse trains 602, 604 has a different amplitude. Optionally, the pulse trains 602, 604 may correspond to an NS signal. In at least one embodiment, the pulse trains 602, 604 may define a spinal cord stimulation therapy and a tissue of interest may represent a portion of a dorsal column. Additionally or alternatively, the pulse trains 602, 604 may correspond to a deep brain stimulation therapy.

The first and second pulse trains 602 and 604 are interleaved with respect to each other such that a series of pulses 608 and 609, respectively, do not overlap one another. For example, the pulses 608 and 608 may be delivered mutually exclusive of one another (e.g., occur at non-overlapping distinct periods of time). Thereby, a current pulse 608a of the first pulse train 602 does not occur during a pulse 609a of the second pulse train 604. The first pulse train 602 includes a series of pulses 608 separated by inter-pulse gaps 622-624 and the second pulse train 604 includes a series of pulses 609 separated by interposes gaps 620. The first and second pulse trains 602 and 604 interleaved in a multiplex manner such that the series of pulses 608 temporally align with the inter-pulse gaps 620, and the series of pulses 609 temporally align with the inter-pulse gaps 622-624. For example, during a period 618, pulses 609a-b of the pulse train 604 are delivered by the IPG 150 separated by the inter-pulse gap 620 approximately the same width as the pulse width of the pulse 608a, such that, each alternating pulse 609 is preceded and/or followed by the inter-pulse gap 620. For example, the pulse width of the pulses 609 may be ten microseconds and the inter-pulse gap 620 may have a length of ten microseconds. It should be noted that in other embodiments, the pulse width and inter-pulse gaps may be greater than (e.g., twenty microseconds, fifty microseconds) or less than (five microseconds, one microsecond) ten microseconds. For example, the pulse widths of the pulses 608, 609 may be between one and fifty microseconds.

The pulse 608a of the pulse train 602 is delivered by the IPG 150 interposed between the inter-pulse gaps 622-624, each having approximately the same width as the pulse width of the pulses 609a-b, respectively, such as ten microseconds. The inter-pulse gaps 622-624 are shown temporally aligned with the pulses 609, and the inter-pulse gaps 620 are shown temporally aligned with the pulses 608 such that no two pulses 608, 609 occur simultaneously. During the inter-pulse gaps 620-624, the first and second pulse trains 602, 604 maintain a neutral or inactive state (e.g., near zero amplitude). For example, during the inter-pulse gaps 620 the segmented electrode 211b may not emit current or voltage. Similarly, during the inter-pulse gaps 622-624, the segmented electrode 211a may not emit a current or voltage.

Based on the properties of the cell membrane (e.g., neuronal cell) of the tissue of interest, as described in connection FIGS. 5a-b, when the membrane of the cell is stimulated by the pulse trains 602 and 604, the membrane integrates the pulse trains 602 and 604 together. The integration by the membrane of the pulse trains 602 and 604 allow the cell to be simultaneously stimulated by the pulse trains 602 and 604 even though the pulses 608 and 609 of the pulse trains 602 and 604 are offset (e.g., time multiplexed).

The first pulse train 602 may be repeated over a set period 612. And the second pulse trains 604 may be repeated over a set period 614. Optionally, the IPG 150 may be programmed or configured to deliver a recharge pulse (e.g., the recharge pulses 603, 605) following at least one of the first and second pulse trains 602 and 604. The recharge pulse has a polarity different than a polarity of the at least one of the first and second pulse trains 602, 604 to provide charge balance. For example, after the pulse train 602 and 604, recharge pulses 603, 605 are delivered by the IPG 150. The recharge pulses 603, 605 have a different polarity than the series of pulse trains 602, 604 to provide a charge balance. The recharge pulses 603, 605 allow the first and second pulse trains 602, 604 to be charge balanced such that a voltage potential across the blocking capacitors for the segmented electrodes 211a and 211b are approximately the same before and after the first and second pulse trains 602, 604 are discharged from the blocking capacitors. For example, an area of charge 626 (e.g., integral of the recharge pulse 603) bounded by the recharge pulse 603 will be approximately the same as areas of charge (not shown) bounded by the pulses 608 of the first pulse train 602. It should be noted, in at least one embodiment, the recharge pulses 603, 605 may occur before the pulse trains 602, 604. Optionally, the recharge pulses 603, 605 may be before and/or after a series of pulse trains 602, 604.

FIG. 7 illustrates a cross-section of the lead 110 at a target position proximate to the tissue of interest. Once the IPG 150 delivers the first and second pulse trains 602 and 604 to the segmented electrodes 211a-b, the segment electrodes 211a-b emit the first and second pulse trains 602 and 604 to form an activation current density (ACD) distribution 702. The ACD distribution 702 is generally distributed between the segmented electrodes 211a-b based on the first and second pulse trains 602, 604. The ACD distribution 702 represents a region within the tissue of interest that experiences local current densities at each point (or a substantial majority of the points) therein, where the local current density is sufficient to achieve activation of the associated local tissue (e.g., neurons, brain matter, etc.) The ACD distribution 702 may represent an area having various shapes and may extend various distances in different directions from a surface of the lead 110. The ACD distribution 702 is steered (e.g., by defining the shape and the direction) to overlay at least a portion of the tissue of interest. For example, the segmented electrode 211a emits current and/or voltage pulses based on the first pulse train 602 delivered by the IPG 150 forming a portion 704 of the ACD distribution 702. Similarly, the segmented electrode 211b emits current and/or voltage pulses based on the second pulse train 604 delivered by the IPG 150 forming a portion 706 of the ACD distribution 702.

The size and/or shape of the ACD distribution 702 and/or the distance to which the ACD distribution 702 extends from the lead 110 may be increased, decreased, and/or steered by adjusting one or more parameters that define the first and second pulse trains 602 and 604, such as the amplitude of the pulses 608, 609 of the pulse trains 602 and 604. For example, the IPG 150 may be configured or programmed to control the parameters (e.g., amplitude, pulse width, or the like) that define a waveform of at least one of the first and second pulse trains 602, 604 in connection with steering the ACD distribution 702 to overlay the tissue of interest. It should be noted that as the pulses 608, 609 emitted by the segmented electrodes 211a-b traverse through the tissue surrounding the lead 110, the amplitude of the resulting electrical potential decreases due to the impedance of the surrounding tissue. The change in the pulse amplitude of one or both of the pulse trains 602 and 604 delivered to the segmented electrodes 211a-b may reduce the effectiveness of the pulse trains 602, 604 in stimulating the tissue of interest.

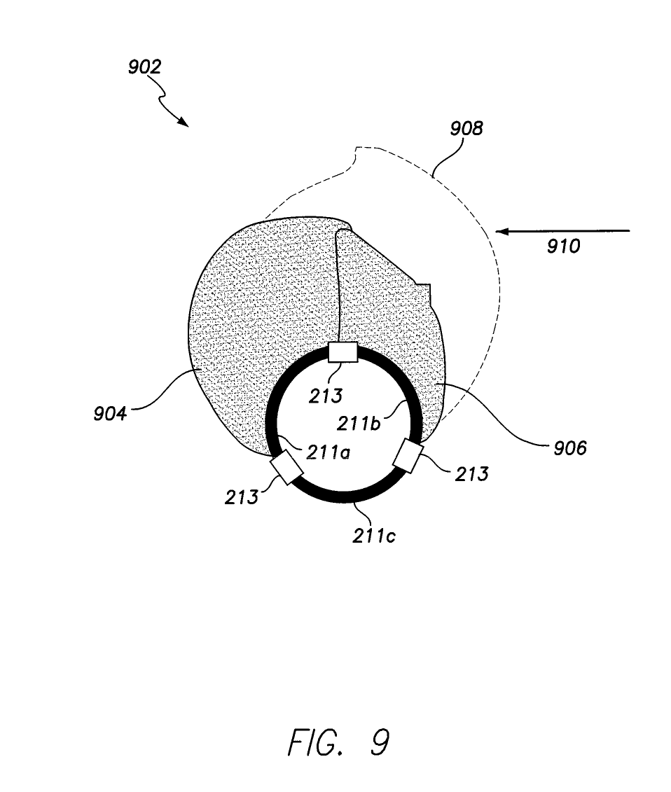

In at least one embodiment, the ACD distribution 902 may be steered in a select direction (relative to the electrodes 211 and/or the lead 110) by adjusting at least one of an amplitude of the pulse trains 602, 804 or a pulse width of the pulse trains 602, 804. For example, the amplitude of one or both of the pulse trains 602 and 604 may be used to direct or steer an ACD distribution 902 as described in connection to FIGS. 8 and 9. FIG. 8 illustrates graphical representations of the series of pulse trains 602 and an adjusted series of pulse trains 804. The series of pulse trains 602 and 804 are delivered to and emitted by the segmented electrodes 211a, 211b, respectively. The adjusted series of pulse trains 804 have a decreased amplitude relative to the series of pulse trains 604. For example, the series of pulses 809 which form the pulse train 804 has a decreased amplitude relative to the series of pulses 609 forming the pulse train 604. After the pulse train 804, an adjusted recharge pulse 805 is delivered by the IPG 150. The adjusted recharge pulse 805 has a decreased amplitude relative to the recharge pulse 605 corresponding to the decrease in the amplitude of the pulses 809.

FIG. 9 illustrates a cross-section of the lead 110 at a target position proximate to the tissue of interest. The ACD distribution 902 is formed by the pulse train 602 and the adjusted pulse train 904. For example, the segmented electrode 211a emits current and/or voltage pulses based on the first pulse train 602 delivered by the IPG 150 forming a portion 904 of the adjusted ACD distribution 902. Similarly, the segmented electrode 211b emits current and/or voltage pulses based on the adjusted pulse train 804 delivered by the IPG 150, thereby forming a portion 906 of the ACD distribution 902. The size and/or shape of the portion 906 of the ACD distribution 902 is smaller, relative to the portion 706, corresponding to the reduction in amplitude of the pulses 809 relative to the pulses 609. The amplitude of the pulses 809, relative to the pulses 609, shifts or changes a direction of the ACD distribution 702 in the direction of an arrow 910.

It should be noted in other embodiments the ACD distribution 702 may be adjusted in various other directions based on a directional ratio of the amplitudes between the pulse trains 602, 604 delivered to the segmented electrodes 211a-b. Optionally, the directional ratio may correspond to an amplitude of a first pulse train (e.g., the pulse train 602) with respect to an amplitude of a second pulse train (e.g., the pulse train 604). For example, the IPG 150 may be programmed or configured to determine a ratio of the first and second amplitudes to steer the ACD distribution 702 toward the tissue of interest. Additionally or alternatively, the ratio may be used by the controller 151 to determine the amplitude for one of the pulse trains. For example, the directional ratio may be two, such that the pulses 608 within the pulse train 602 delivered by the IPG 150 may be configured to be twice the size of the pulses (e.g., the pulses 809) for the pulse train (e.g., adjusted pulse train 808) delivered by the IPG 150 to the other electrode. In another example, the directional ratio may be zero point five, such that the pulses 608 within the pulse train 602 delivered by the IPG 150 may be configured to be half the size of the pulses (e.g., the pulses 609) for the pulse train (e.g., pulse train 608) delivered by the IPG 150 to the other electrode. It should be noted that the above ratios are exemplary, in other embodiments the ratio may be greater than or less than zero point five. Additionally or alternatively, the directional ratio may be used to indicate a direction of the ACD distribution. For example, a ratio of greater than one may indicate that the ACD distribution is directed towards the first pulse train. In another example, a ratio less than one may indicate that the ACD distribution is directed towards the second pulse train.

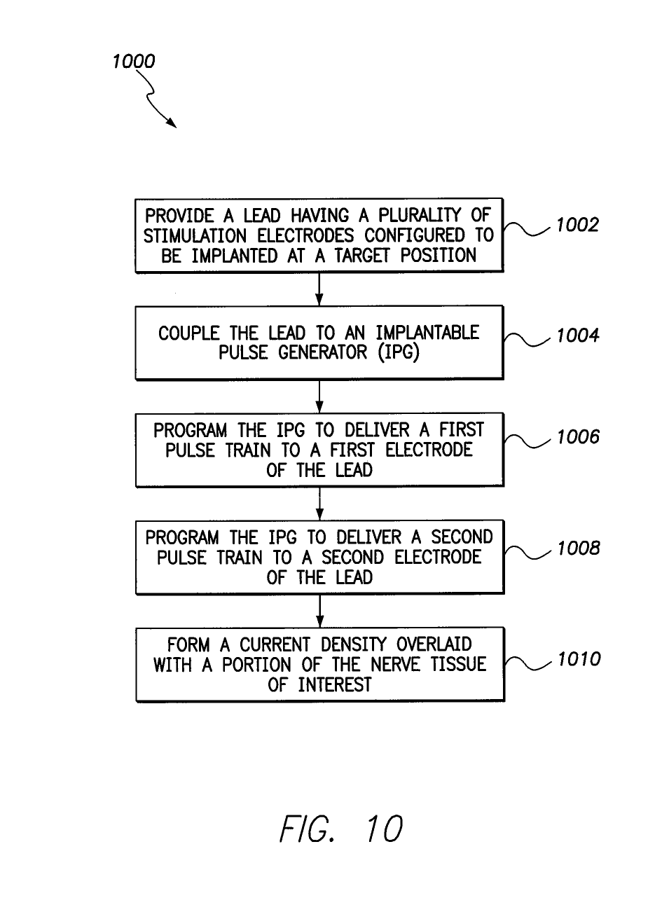

FIG. 10 is a flowchart illustrating a method 1000 for current steering a neurostimulation signal. The method 1000, for example, may employ structures or aspects of various embodiments (e.g., systems and/or methods) discussed herein. For example, an implantable pulse generator (IPG) may be similar to the IPG 150 (FIG. 1) or may include other features, such as those described or referenced herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. Furthermore, it is noted that the following is just one possible method for current steering a neurostimulation signal. It should be noted, other methods may be used, in accordance with embodiments herein.

One or more methods may (i) provide a lead coupled to an implantable pulse generator (IPG), (ii) program the IPG to deliver at least a first pulse train to a first electrode and a second pulse train to a second electrode, and (iii) forming an activation current density (ACD) distribution overlaid with at least a portion of the tissue of interest.

Beginning at 1002, the lead 110 may be provided that has a plurality of stimulation electrodes (e.g., the array of electrodes 111, the segmented electrodes 211, the ring electrodes 212) configured to be implanted at a target position. The target position may be proximate to the tissue of interest (e.g., spinal cord tissue, dorsal column, portions of the brain, or the like).

At 1004, the lead 110 may be coupled to the IPG 150. For example, the terminals of one or more leads 110 are inserted within the IPG header of the IPG 150 for electrical connection with respective connectors. Pulses are generated by the IPG 150 and are conducted through the IPG header to conductors of the lead 110 and applied to tissue of interest (e.g., nerve cells, brain cells, brain matter) of a patient via stimulation electrodes 111.

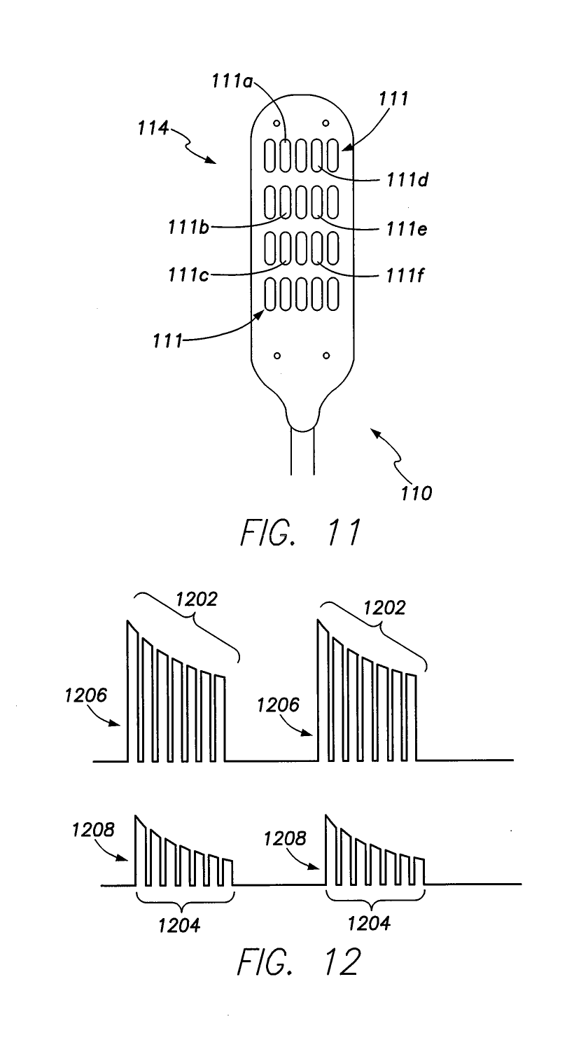

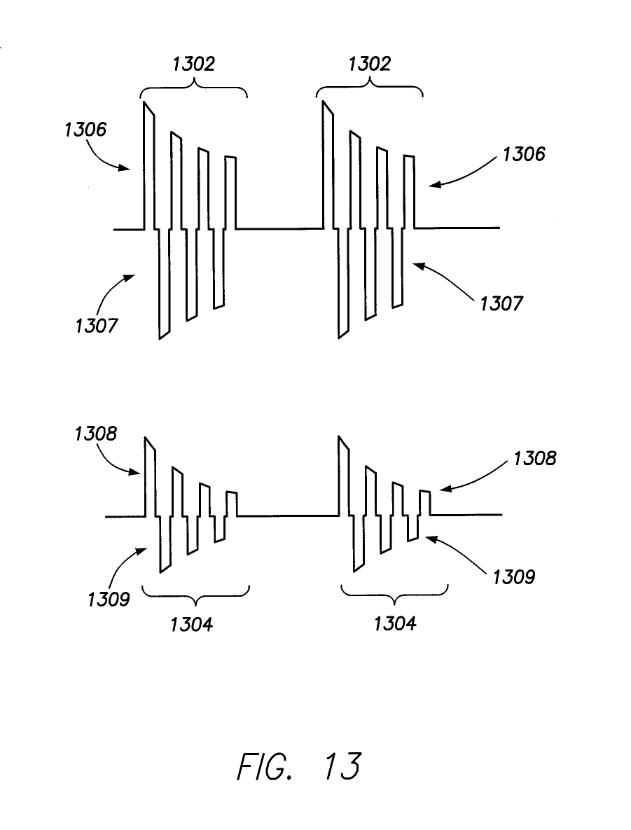

At 1006, the IPG 150 may be programmed to deliver a first pulse train 1202 to a first electrode 111b of the lead 110. For example, the IPG 150 may be programmed or receive stimulation programs from the controller device 160. The stimulation program may include pulse specifications or parameters (e.g., amplitude, frequency, pulse width, number of pulses 1206) of the pulses 1206 that form the first pulse train 1202, as shown in FIG. 12, and delivered by the IPG 150 forming a part of the NS signal. FIG. 11 is a proximate view of the paddle structure 114 from an embodiment of the lead 110. The IPG may be programmed to deliver the first pulse train 1202 to the first electrode 111b of the lead 110.

At 1008, the IPG may be programmed to deliver a second pulse train 1204 to a second electrode 111e of the lead 110. The first and second pulse trains 1202 and 1204 may be temporally offset with respect to each other such that the pulses 1206 of the first pulse train 1202 are interleaved with or do not occur during a pulse 1208 of the second pulse train 1204. The IPG 150 may be programmed or receive stimulation programs from the controller device 160. The stimulation program may include pulse specifications or parameters (e.g., amplitude, frequency, pulse width, number of pulses 1208) of the pulses 1208 that form the first pulse train 1204. Optionally, the pulse specification may include a direction ratio indicating a value of the amplitude of the pulses 1208 forming the second pulse train 1204 with respect to the amplitude of the pulses 1206 forming the first pulse train 1202.

In at least one embodiment, the pulse specifications may include recharge pulses within a series of pulse trains 1302, 1304 to maintain a charge balance such that at least one of the pulses 1307, 1309 within the pulse train 1302, 1304 is a different polarity. For example, the IPG 150 may be configured or programmed to deliver, within the first pulse train (e.g., the pulse train 1302), a first pulse 1306a having a positive polarity and a second pulse 1307b having a negative polarity to provide charge balance. FIG. 15 illustrates graphical representations of pulse trains 1302, 1304 delivered to and emitted by the segmented electrodes 111b, 111e, respectively, shown in FIG. 11. The pulse trains 1302 1304 are formed from a series of pulses 1306-1309 corresponding to a state of the electrodes 111b and 11e. For example, during the pulses 1306 the electrode 111b may be in an anode state and the electrodes 111a and 111c may be in a cathode state. Additionally, during the pulses 1307, the polarity of the electrodes 111a-c may be changed by the switching circuitry 157 in order to maintain charge balance. For example, during the pulses 1307 the electrode 111b may be in the cathode state and the electrodes 111a and 111c may be in the anode state. Similarly, during the pulses 1308 the electrode 111e may be in an anode state and the electrodes 111d and 111f may be in a cathode state. During the pulses 1309, the polarity of the electrodes 111d-f may be changed by the switching circuitry 157, switching the electrode 111e to the cathode state and the electrodes 111d and 111f to the anode state. It should be noted that in other embodiments, a separate recharge pulse may be delivered by the IPG 150 before and/or after the pulse trains 1202, 1204.

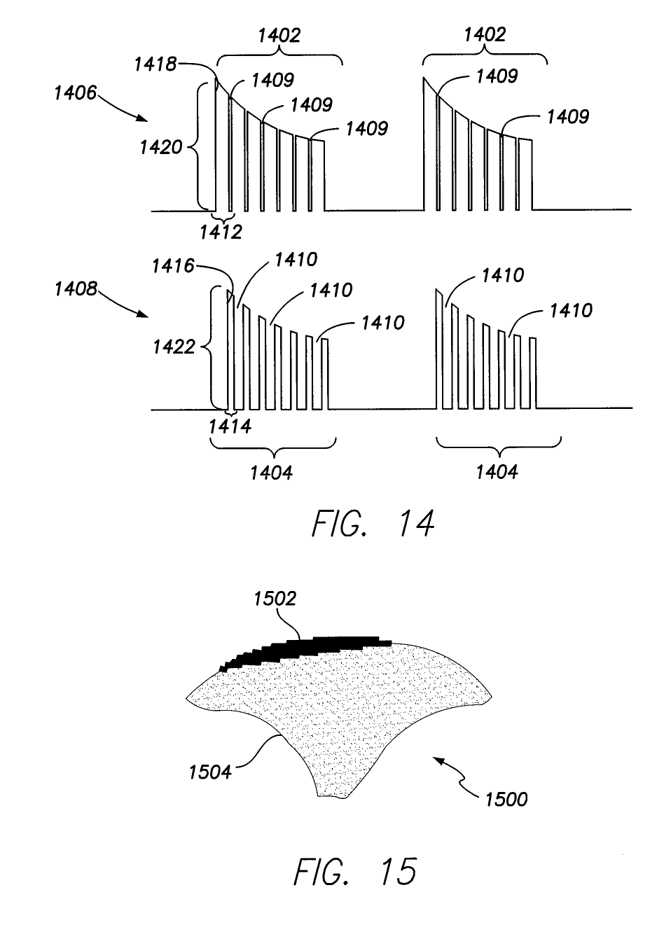

It should be noted that in other embodiments the duty cycle of the pulse trains may be greater than or less than 50%. Additionally or alternatively, the duty cycles of the pulse trains may not be the same to steer the ACD distribution. For example, the duty cycle of the pulse trains may be based on a directional ratio. Additionally or alternatively, the IPG 150 may be configured or programmed to determine the ratio of the first and second pulse widths to steer the ACD distribution toward the tissue of interest. FIG. 14 illustrates graphical representations of pulse trains 1402, 1404 delivered to and emitted by the electrodes 111b, 111e, respectively, shown in FIG. 11. The directional ratio may correspond to a duty cycle of a first pulse train (e.g., the pulse train 1402) with respect to a duty cycle of a second pulse train (e.g., the pulse train 1404). The ratio may be used by the controller 151 to determine the duty cycle or pulses width of another pulse 1406, 1408. For example, the directional ratio may be zero point two five, such that the pulse train 1402 may have a duty cycle of 80%. Based on the directional ratio, the controller 151 may determine that the pulse train 1404 may have a duty cycle of 20%, or twenty five percent of the duty cycle of the pulse train 1402. The duty cycles of the pulse trains 1402, 1404 correspond to pulse widths 1412, 1414 of the pulses 1406, 1408 that form the pulse trains 1402, 1404. It should be noted that the above directional ratio is exemplary, in other embodiments the ratio may be greater than or less than zero point two five.

The pulse trains 1402, 1404 are interleaved with respect to each other such that the pulses 1406 forming the pulse train 1402 do not occur during the pulses 1408 that form the pulse train 1404. For example, the pulse width 1412 of the pulses 1406 may be approximately four microseconds, and the pulse width 1414 of the pulses 1408 may be approximately one microsecond. The pulses 1406 and 1408 are separated by inter-pulse gaps 1409 and 1410, respectively. A length of the inter-pulse gap 1409 is approximately the same as the pulse width of the pulses 1408. Similarly, a length of the inter-pulse gap 1410 is approximately the same as the pulse width of the pulses 1406.

As described above, based on the properties of the cell membrane (e.g., neuronal cell), as described in connection with FIGS. 5a-b, when the membrane of the cell is stimulated by the pulse trains 1402 and 1404, the membrane integrates the pulse trains 1402 and 1404 together. FIG. 15 illustrates a graphic 1500 of an activation area 1502 resulting from the pulse trains 1402 and 1404 emitted by the electrodes 111b and 111e of a dorsal column 1504. The activation area 1502 may correspond to an area that is overlaid with and/or stimulated by the ACD distribution formed by the pulse trains 1402 and 1404. For example, the pulse trains 1402 and 1404 may correspond to an NS signal configured for a spinal cord stimulation therapy. It should be noted that in other embodiments the NS signal may be configured for deep brain stimulation, peripheral nerve stimulation, and the like. The ACD distribution may be configured by the controller 151 to direct the NS towards the tissue of interest within the dorsal column 1504 such that the ACD distribution is overlaid with the dorsal column 1504. For example, the IPG 150 may be programmed or configured to steer the ACD distribution toward the tissue of interest within the dorsal column 1504.

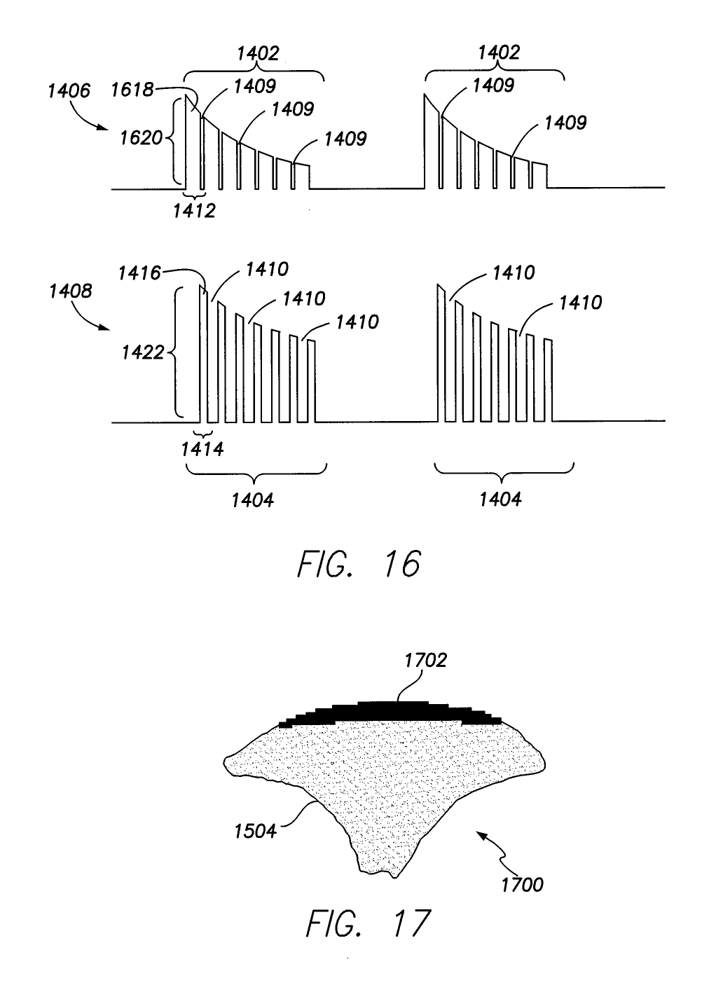

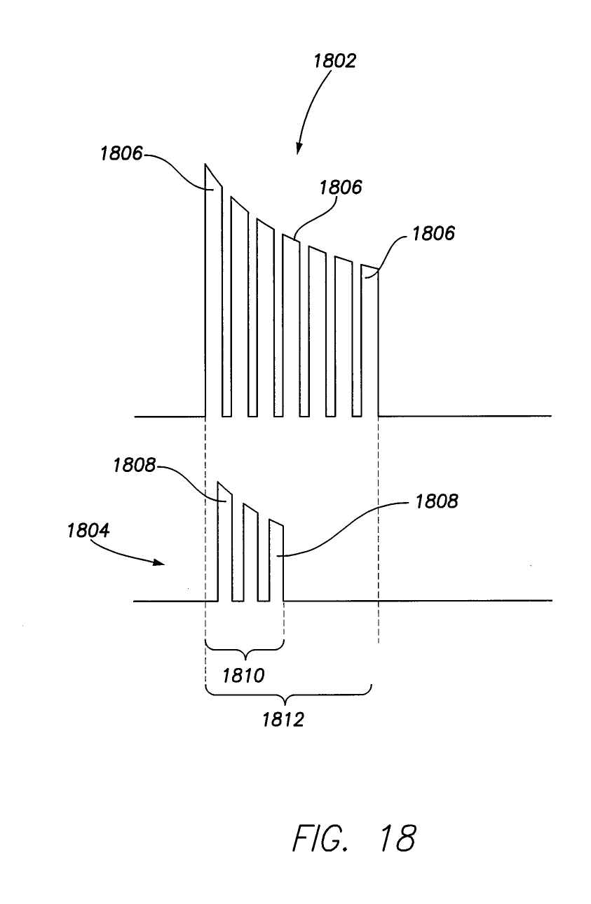

It should be noted in various embodiments the amplitudes 1620 and 1422 and duty cycles of the pulse trains 1402, 1404 may be different. FIG. 16 illustrates graphical representations of the pulse trains 1402, 1404 shown in FIG. 14. The pulse train 1402 has an adjusted amplitude 1620 relative to the amplitude 1420 shown in FIG. 14. The adjusted amplitude 1620 reduces an area of charge 1418 (e.g., integral of the pulses 1406) bounded by the pulses 1406 to the area of charge 1618. The area of charge 1618, based on the adjusted amplitude 1620, may be approximately the same as an area of charge 1416 bounded by the pulses 1408 of the pulse train 1402. FIG. 17 illustrates a graphic 1700 of an activation area 1702 resulting from the NS signal emitted by the electrodes 111b and 111e of a dorsal column 1504 corresponding to the pulse trains 1402 and 1404 of FIG. 16. Based on the area of charges 1618 and 1416 being approximately equal, the activation area 1702 has shifted position with respect to the activation area 1502. The activation area 1702 is at an equidistant position of the dorsal column 1504 corresponding to a position between the electrodes of 111b and 111e.

In at least one embodiment the lengths 1810, 1812 of the pulse trains 1802, 1808, respectively, may not be the same. The tissue of interest may comprise or include a first and second types of cells having first and second chronaxie, respectively. The IPG 150 may be programmed or configured to determine a length of the first pulse train (e.g., the pulse train 1802) based on the first chronaxie and a length of the second pulse train (e.g., the pulse train 1808) based on the second chronaxie. For example, a length 1802 of a first pulse train 1802 may be based on a chronaxie of cells forming a first subset of the tissue of interest and a length 1812 of a second pulse train 1808 may be based on a chronaxie of another set of cells forming a second subset of the tissue of interest. FIG. 18 illustrates graphical representations of pulse trains 1802, 1804 delivered to and emitted by the segmented electrodes 111b, 111e, respectively, shown in FIG. 11. The chronaxie, generally, may represent the amount of charge delivered over a time period, provided by the pulse train 1802, 1808 (e.g., area of the pulses 1806, 1808), that is needed to stimulate the tissue of interest. Particularly, chronaxie is the minimum time required for an electric current double the strength of the rheobase to stimulate a muscle or a neuron. Rheobase is the lowest intensity with indefinite pulse duration which can stimulate muscle or neuronal cells. For example, the IPG 150 may be programmed to determine or receive instructions or parameters to stimulate a tissue of interest that includes two types of neurons or cells, each having a different chronaxie. Each of the pulse trains 1802, 1808 may be configured to stimulate one set of the neurons. Based on the chronaxie levels, the neurons stimulated by the pulse train 1804 may have a shorter chronaxie than the neurons stimulated by the pulse train 1802. Based on the shorter chronaxie, the length 1810 of the pulse train 1804 may be shorter than the length 1812 and corresponds to when the chronaxie or stimulation of the neurons is reached.

At 1210, the ACD distribution is formed and overlaid with a portion of the tissue of interest.

Optionally, a response may be measured from the tissue of interest. The controller 151 may compare the measured response with a predetermine threshold to determine whether the tissue of interest has been stimulated by the NS signal (e.g., the pulse trains 602, 604), and instruct the IPG 150 to stop delivery of the pulse trains based on the measured response. For example, evoked compound activation potential (ECAP) signals may be generated by neuronal transmembrane currents of neurons activated following or in response to the pulse trains 602, 604 emitted by one or more of the electrodes 111. The electrodes 111 may also be configured to acquire electrical potential measurements (e.g., voltage, current) or electrical signals for the sensory circuit 158, such as the ECAP signals. The controller 151 may compare the measured ECAP signals with a predetermined threshold stored on the memory 161 to indicate when the tissue of interest has been stimulated based on whether the measured ECAP signal is above the predetermined threshold. Once the controller 151 determines that the tissue of interest has been stimulated, the IPG 150 may stop delivery of the pulse trains 602, 604.

The controllers 151, 206 and the controller device 160 may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the controllers 151, 206, 1006 and the controller device 160 may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The controllers 151, 206, and the controller device 160 may execute a set of instructions that are stored in one or more storage elements, in order to process data. The storage elements may also store data or other information as desired or needed. The storage element may be in the form of an information source or a physical memory element within the controllers 151, 206, and the controller device 160. The set of instructions may include various commands that instruct the controllers 151, 206, and the controller device 160 to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112(f), unless and until such claim limitations expressly use the phrase “means for” followed by a statement of function void of further structure.

What is claimed is:

1. A method for current steering a neurostimulation signal delivered by an implantable pulse generator (IPG) between a plurality of electrodes coupled to the IPG toward tissue of interest, the method comprising:
programming the IPG to deliver at least a first pulse train to a first electrode and a second pulse train to a second electrode, wherein the first and second pulse trains are interleaved with one another such that the first and second pulse trains form an activation current density (ACD) distribution steered to overlay the tissue of interest.

2. The method of claim 1, further comprising programming the IPG to control parameters that define a waveform of at least one of the first and second pulse trains in connection with steering the ACD distribution to overlay the tissue of interest.

3. The method of claim 1, wherein the first pulse train includes first pulses separated by first gaps and the second pulse train includes second pulses separated by second gaps, the first and second pulse trains interleaved in a multiplex manner such that the first pulses temporally align with the second gaps and the second pulses temporally align with the first gaps.

4. The method of claim 1, further comprising programming the IPG to steer the ACD distribution in a select direction by adjusting at least one of an amplitude or a pulse width of the first and second pulse trains.

5. The method of claim 1, wherein the first and second pulse trains define a spinal cord stimulation therapy and the tissue of interest represents a portion of a dorsal column; the method further comprising programming the IPG to steer the ACD distribution toward the tissue of interest within the dorsal column.

6. The method of claim 1, further comprising programming the IPG to:
deliver pulses of the first pulse train with a first amplitude and pulses of the second pulse train with a second amplitude; and
determine a ratio of the first and second amplitudes to steer the ACD distribution toward the tissue of interest.

7. The method of claim 1, further comprising programming the IPG to:
deliver pulses of the first pulse train with a first pulse width and pulses of the second pulse train with a second pulse width; and
determine a ratio of the first and second pulse widths to steer the ACD distribution toward the tissue of interest.

8. The method of claim 5, wherein the first and second pulse are between one microsecond and fifty microseconds.

9. The method of claim 1, wherein the first and second electrodes are at least one of segmented ring electrodes, electrodes on a paddle structure of the lead, or ring electrodes.

10. The method of claim 1, wherein the first and second pulse trains correspond to a deep brain stimulation therapy.

11. The method of claim 1, further comprising programming the IPG to deliver the first and second pulse trains with the same polarity.

12. The method of claim 1, further comprising programming the IPG to deliver a recharge pulse following at least one of the first or second pulse trains, wherein the recharge pulse has a polarity different than a polarity of the at least one of the first or second pulse trains to provide charge balance.

13. The method of claim 1, wherein the tissue of interest comprises first and second types of cells having first and second chronaxie, respectively, further comprising programming the IPG to determine a length of the first pulse train based on the first chronaxie and a length of the second pulse train based on the second chronaxie.

14. The method of claim 1, further comprising programming the IPG to deliver, within the first pulse train, a first pulse having a positive polarity and a second pulse having a negative polarity to provide charge balance.

15. A system for current steering a neurostimulation signal comprising:
a lead configured to be implanted at a target position proximate to or within a tissue of interest;
a plurality of electrodes on a surface of the lead; and
an implantable pulse generator (IPG) coupled to the lead, the IPG configured to deliver at least a first pulse train to a first electrode and a second pulse train to a second electrode, wherein the first and second pulse trains are interleaved with one another such that the first and second pulse trains form an activation current density (ACD) distribution steered to overlay the tissue of interest.

16. The system of claim 15, wherein the IPG is further configured to control parameters that define a waveform of at least one of the first and second pulse trains in connection with steering the ACD distribution to overlay the tissue of interest.

17. The system of claim 15, wherein the first pulse train includes first pulses separated by first gaps and the second pulse train includes second pulses separated by second gaps, the first and second pulse trains are interleaved in a multiplex manner such that the first pulses temporally align with the second gaps and the second pulses temporally align with the first gaps.

18. The system of claim 15, wherein the IPG is further programmed to steer the ACD distribution in a select direction by adjusting at least one of an amplitude or a pulse width of the first and second pulse trains.

19. The system of claim 15, wherein the first and second pulse trains define a spinal cord stimulation therapy and the tissue of interest represents a portion of a dorsal column, wherein the IPG is further configured to steer the ACD distribution toward the tissue of interest within the dorsal column.

20. The system of claim 15, wherein the first and second pulse trains correspond to a deep brain stimulation therapy.

21. The system of claim 15 wherein one or more of the first electrode and the second electrode comprise segmented ring electrodes and the IPG being further programmed to steer the ACD distribution in a select direction by delivering the first and second pulse trains to selected segments of the first and second electrodes.
